(19) United States
(12) Patent Application Publication
(10) Pub. No.: US 2016/0030302 A1
(43) Pub. Date: Feb. 4, 2016

(54) Method and device for the surface working of a cosmetic product

(71) Applicant: CHROMAVIS S.p.A., MILANO (IT)

(72) Inventors: Nicolò LARCERI, MILANO (IT); Fabio MORONI, Cremona (IT)

(21) Appl. No.: 14/809,332

(22) Filed: Jul. 27, 2015

(30) Foreign Application Priority Data
Jul. 31, 2014 (IT) MI2014A001406

(51) Int. Cl. A61K 8/02; A61Q 19/00; B23B 13/04; A45D 33/18; B23B 5/00

(52) U.S. Cl. CPC A61K 8/0216; A45D 33/18; B23B 5/00; B23B 13/04; A61Q 19/00; B23B 2220/24; B23B 2226/00; B23B 2228/28; A61K 2800/805

(57) ABSTRACT

A surface working method of a cosmetic make-up product comprising the step of setting the cosmetic product in rotation about an axis of symmetry thereof and, simultaneously, moving a cutting tool above the surface of the cosmetic product so as to remove at least a part of said surface, thus giving the surface a predetermined shape. The finding also relates to a device for implementing said method and a cosmetic product made with such a device.

This application claims the benefit of Italian patent application N. MI2014A001406 filed on Jul. 31, 2014, the disclosure of which is incorporated herein by reference.

### TECHNICAL FIELD

The present invention relates to a surface working method and device of a cosmetic make-up product.

In particular, it relates to a surface working method of a cosmetic product of the baked type, with rounded or flat surface, and to a cosmetic product obtained by such a method.

### BACKGROUND

Currently, cosmetics products for make-up, and specifically of baked products, are produced by depositing a paste on a base. Often more pastes of different colors are deposited in different areas on the same base, so as to obtain a multicolored look on the finished product.

Once the positioning step of the paste(s) on the base has been completed, following a preforming carried out in a die, the whole is dried in the furnace.

After drying, the product surface is cleaned by abrasion with a sponge.

Cleaning with a sponge is intended to remove from the surface the outer layer of the product having a dried or cracked crust after drying. In this way, therefore, most imperfections generated in the previous working steps are removed to obtain a surface of the cosmetic make-up product with a smooth and neat appearance.

The manual cleaning step is very expensive because it takes a long time. Moreover, it must be carried out by very experienced and capable personnel.

In fact, it should be noted that the pressure manually made on a substantially friable surface such as that of the cosmetic make-up product is never homogeneous. Therefore, a lot of practice and skill is required to obtain a quality result through this method.

However, it should be noted that even though the cleaning is carried out by extremely experienced personnel, every cosmetic product, after cleaning, has a shape slightly different from the other. In fact, it is impossible to obtain a homogeneous result manually removing a surface layer of dry paste from the cosmetic product. Moreover, the cosmetic products made using this technique also differ in the weight, in addition to shape. In fact, the manual working is unable to ensure a homogeneous removal of the excess cosmetic product to obtain a certain weight.

This creates problems of packaging of the finished product, which, as known, takes place in containers (usually plastic) obtained by molding, and therefore, all perfectly identical. There is therefore the possibility that some cosmetic products must be discarded as they are too low or too high to be accommodated in the final containers. Many products are also discarded because their weight is less than a certain limit.

Another problem that occurs, especially in multicolor cosmetic products, is that the manual cleaning operation using a sponge slightly mixes the colors on the surface, thus making the final product lose definition.

### SUMMARY

The object of the present invention is to provide a surface working method and device of a cosmetic make-up product which overcomes the mentioned prior art drawbacks.

A further object of the present invention is to reduce the working time of the cosmetic products, thereby minimizing the costs thereof.

Yet another object of the invention is to provide a working method which makes the shape and the final weight of the cosmetic products for make-up obtained therewith more uniform, so as to easily package them in standardized containers, thus avoiding waste.

Last but not least, an object of the invention is to provide a surface working method that allows obtaining an excellent definition on a multicolor product.

These and other objects are achieved by a surface working method and device of a cosmetic make-up product according to the technical teachings of the appended claims.

### BRIEF DESCRIPTION OF THE DRAWING FIGURES

Further features and advantages of the invention will become apparent from the description of a preferred but non-exclusive embodiment of the device, shown by way of a non-limiting example in the accompanying drawings, in which:

FIG. 1A is a perspective view of a surface working device of a cosmetic product according to the present description;

FIG. 1B is a view of an enlarged detail of the device in FIG. 1;

FIG. 2 is a front view of the device in FIG. 1;

FIG. 3 is a side view of the device in FIG. 1;

FIGS. 4A, 4B and 4C show the sequential movement steps of some cosmetic products through various working positions of the device in FIG. 1;

FIGS. 5A and 5B show a working start and end step of a cosmetic product, respectively;

FIG. 6 is a simplified side view of a cosmetic product during working carried out with the device in FIG. 1; and

FIG. 7 is an enlarged view of a part of FIG. 6.

### DETAILED DESCRIPTION

With reference to the above figures, a surface working device of a cosmetic product 2 is shown, indicated as a whole with reference numeral 1.

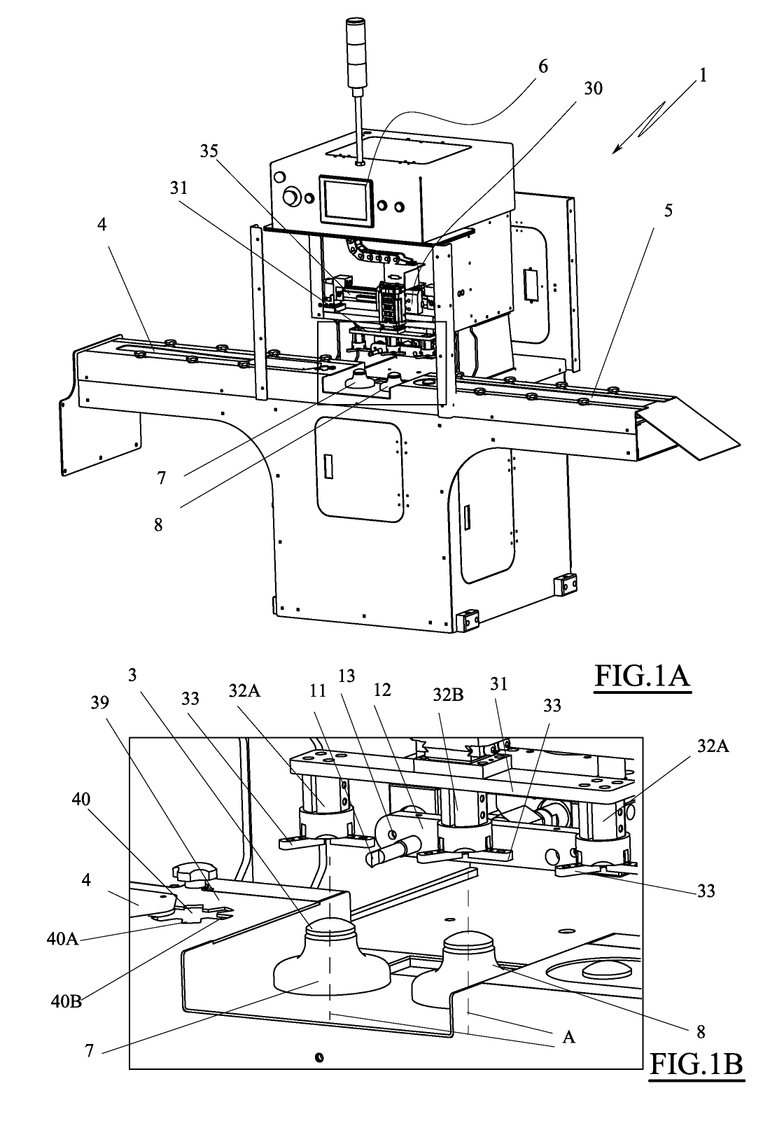

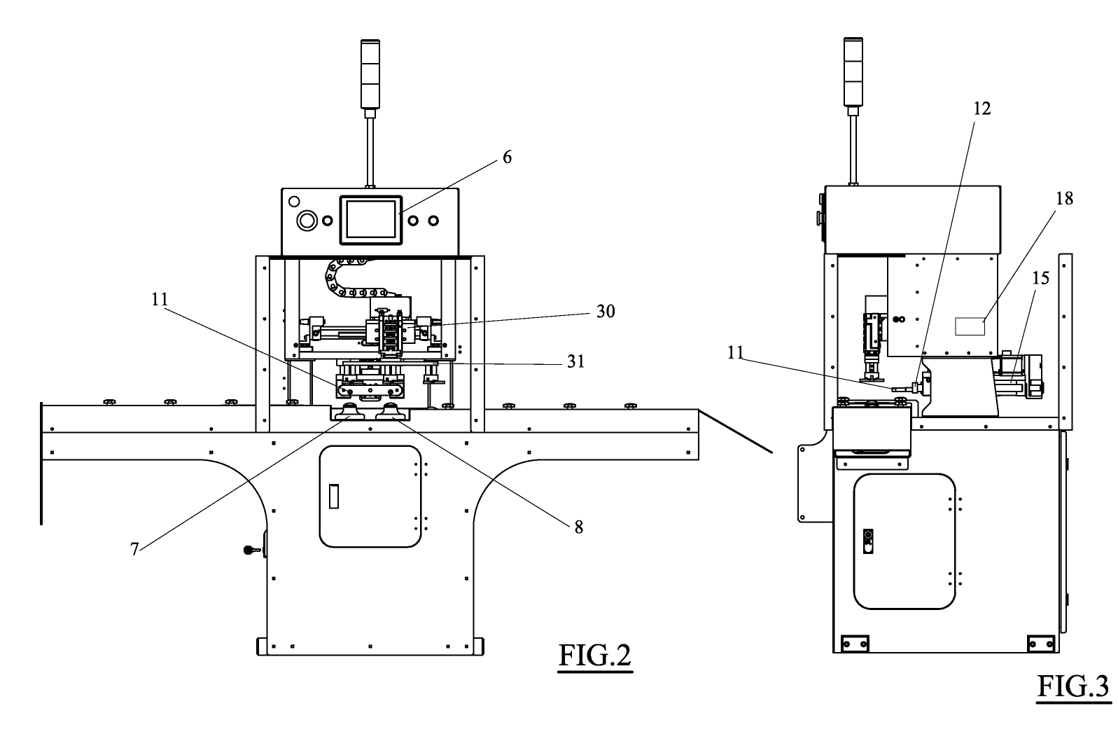

The device, clearly visible in FIG. 1, comprises a frame 3 which supports a loading conveyor belt 4 and an unloading conveyor belt 5. The conveyor belts are driven by suitable motors, not shown, but controlled by a control unit 6.

A first 7 and a second rotating base 8 are provided between the conveyor belts, in a dedicated seat 9 accessible from the outside.

The first base 7 can rotate (driven in rotation by a suitable motor, not shown) about an axis of rotation A, preferably vertical. In particular, it is provided with blocking means 10 of a cosmetic product which is placed on top of it during a working step of the cosmetic product itself. In particular, the cosmetic product is placed with a circular axis of symmetry thereof coincident with axis A.

In the proximity of the first base, as clearly visible in FIG. 1B, a cutting tool 11 is present, associated to movement means adapted to move it along the surface of the cosmetic product at least when said rotating base is in rotation, so as to work the surface of the cosmetic product, removing at least a surface part thereof.

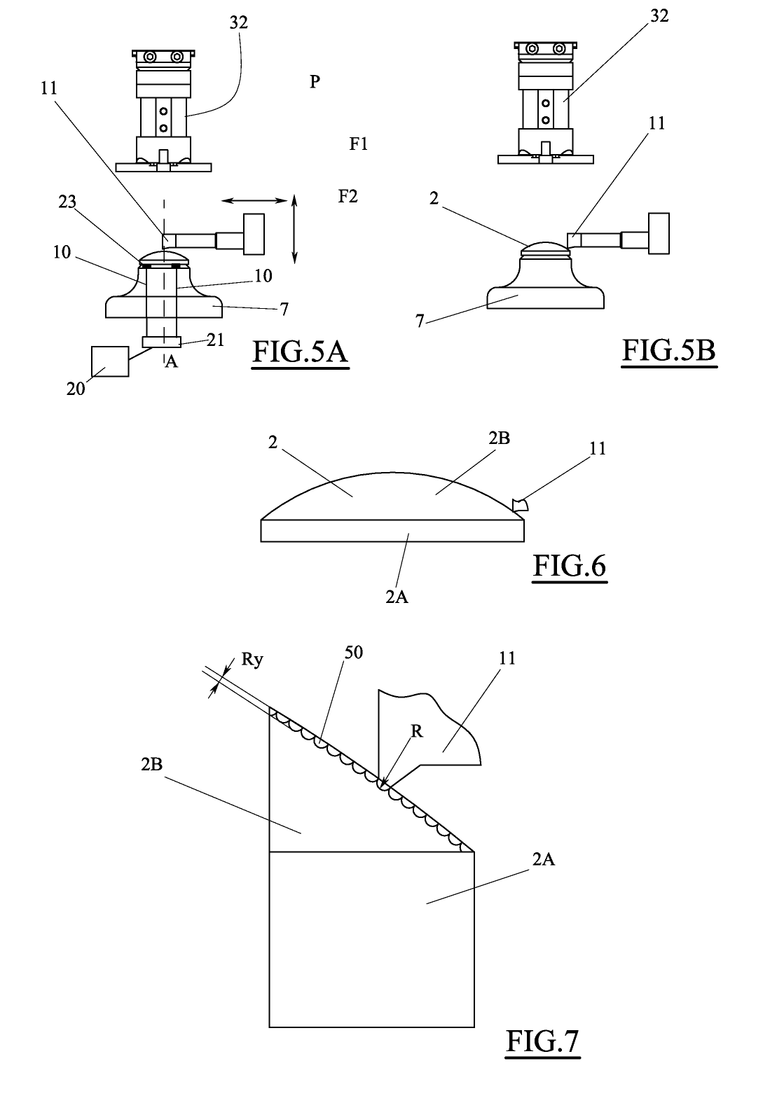

Preferably, the cutting tool 11 is supported by a plate 12 sliding on guides 13 perpendicular to axis A which allow the same to carry out a movement along arrows F1 in FIG. 5A. Such a movement is controlled by the control unit and carried out using a stepper motor 15 visible in FIG. 3.

The stepper motor 15, plate 12 and guides 13 form a block supported by further guides 16 which allow a movement to the cutting tool along direction F2 in FIG. 5A. Such a movement is carried by an actuator 18, simply schematized in FIG. 3 but also controlled by the control unit 6.

In the practice, the movement means described above allow the cutting tool 11 to move under the control of the control unit 6, anywhere above the rotating base 7, in the direction of arrows F1 and F2. The movement of the cutting tool preferably takes place within a plane P, containing the rotation axis A of the base. Therefore the movement of the cutting tool 11 takes place along a trajectory lying in the plane P containing the rotation axis of the rotating base 7, and the axis of symmetry of the make-up cosmetic product.

FIG. 5A schematizes some channels 10 that run through the base and are connected to a vacuum source 20 (e.g. a vacuum pump, a vacuum vessel or any other means adapted to create a suction force sufficient to keep the product in place during its rotation and working), advantageously through valve means 21. Each of channels 10 has an opening placed on the surface of the rotating base 7 to which a sealing means 23 is associated, which preferably has an overturned suction or gasket shape. It is also possible to provide for a single large suction cup that is coupled to the base of the cosmetic product.

As can be seen from the figures, the device comprises a second rotating base 8, structurally identical to the first one, which rotates synchronized with the first one. A blowing nozzle (not shown for simplicity) is advantageously provided above it which blows air onto the surface of the cosmetic product just worked on the first rotating base 7.

Above the first and the second rotating base a carriage 30 is present, sliding on its own guide 35, which supports a cross member to which three manipulators 32A, 32B, 32C are attached, each comprising three mobile fingers 33 for gripping the cosmetic product and moving it between the loading conveyor belt 4, the first 7 and the second rotating base 8 and the unloading conveyor belt 5.

As can be seen in FIG. 1B, the first conveyor belt 4 flows on a loading plane 39 associated with it. It is provided with a star-shaped opening 40 which defines windows 40A, 40B which allow the mobile fingers 33 to grip the cosmetic product, preferably at a base thereof.

To conclude the description it should be said that at least above the first rotating base 7, or at least in the area surrounding it, means of forced aspiration are provided (which are not shown) arranged so as to aspirate the cosmetic dust produced during the working.

The operation of the device described is clear to the man skilled in the art and is substantially as follows.

An operator loads a plurality of cosmetic make-up products 2 on the first conveyor belt.

The cosmetic products for make-up 2 (FIG. 6) have a base 2A on which a make-up product 2B is placed. By way of example, the cosmetic make-up product 2 is of the baked type. The make-up product 2B is a cosmetic paste dried in a furnace and therefore has a surface layer with cracks and imperfections. It should be noted that during the steps before the baking, the cosmetic paste 2B has been deposited on the base in excess to what will make the final cosmetic product. A part of the excess cosmetic paste will be removed through the device described herein.

The first conveyor belt 4 moves bringing the products 2 towards the first rotating base 7. When a cosmetic product is deposited above the star-shaped opening 40, a sensor detects the presence thereof and the conveyor belt is stopped.

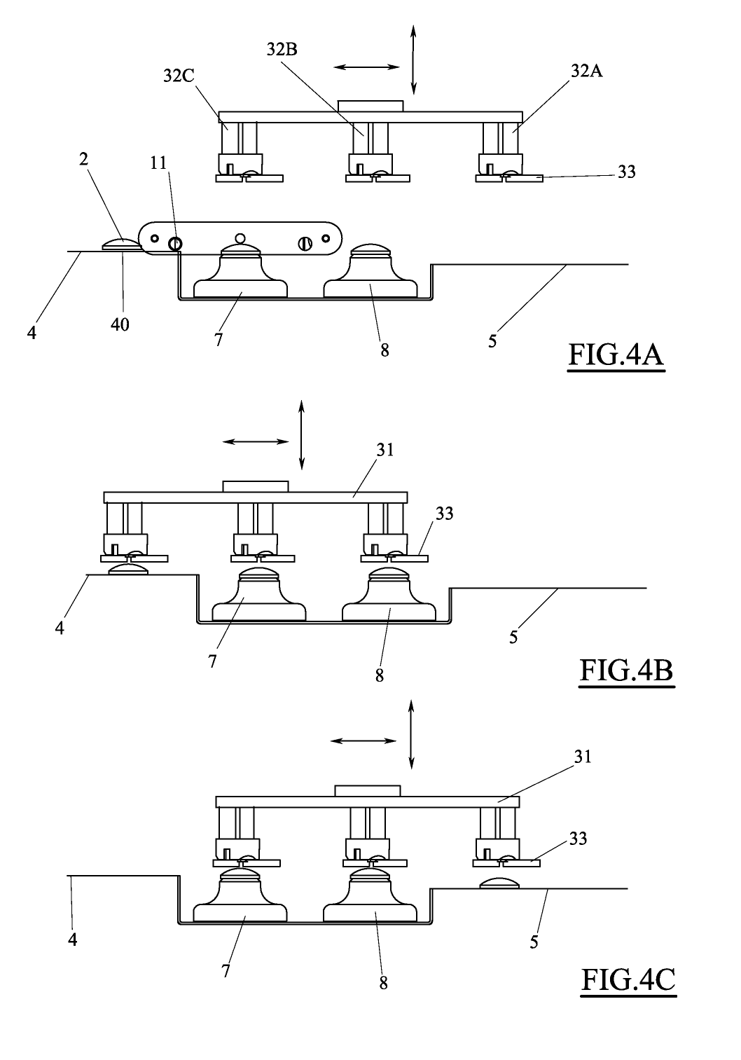

The configuration is now that shown in FIG. 4A, where the cosmetic product placed above the first rotating base 7 is being worked. It should be noted that the working of product 2 and the movement of the first conveyor belt can also be simultaneous for increasing productivity. In any case, they all are regulated in an optimized manner by the control unit 6.

At the end of the working of the product positioned in FIG. 1 above the first rotating base 7, the cross member 31 and therefore all the manipulators, move to the left and go down above the cosmetic products, as shown in FIG. 4B. In this position, fingers 33 of each manipulator tighten around the bases of the three underlying cosmetic products, namely that placed over the star-shaped opening 40, that placed above the first rotating base 7 and placed above the second rotating base 8.

Once the three cosmetic products have been gripped, the cross member 31 rises slightly and moves to the right in FIG. 4B; when each manipulator 32A, 32B and 32c is in position, the cross member lowers and the fingers open, dropping the cosmetic product into position.

In the first rotating base 7, the valve means 21 are opened and channels 10 are thus under depression, so that the cosmetic product is temporarily fixed to the rotating base 7 due to the suction effect.

This also happens with respect to the second rotating base 8.

Meanwhile, the cosmetic product previously present on the second rotating base 8 is now positioned on the second conveyor belt which, by activating, moves it away from the working area.

The rotating bases are operated in rotation, thus dragging also the cosmetic products placed thereon in rotation about an axis A, coincident with that of symmetry of products 2. In the embodiment shown, such an axis is arranged vertically.

During the rotation of the cosmetic product, the cutting tool 11 is moved above the surface thereof so as to remove at least part of said surface, thus giving it a predetermined shape.

FIG. 5 shows that the motion of tool 11 begins in the proximity of axis A and ends at the edge of the base 2A removing, by virtue of the rotational motion of the rotating base, all that part of make-up product that is located above the line defined by the tool trajectory. Actually, the cosmetic product is worked through a turning. The movement of the cutting tool 11 is radial with respect of the cosmetic make up-product 2, and with respect of the rotating base 7.

Advantageously, the cutting tool 11 moves on a single plane P. Its motion may be along a curved trajectory as in the case described, or along a straight trajectory or along any other trajectory, such as wavy.

Moreover, the tool may be moved above the product surface by a fixed distance at each revolution of the cosmetic product (feed).

Such a working creates a groove 50 on the surface of the cosmetic product with a peak-to-trough Ry in the range between and 60 micron. If the movement of the rotating base is synchronized with the movement of the cutting tool 11, such a groove is a spiral. In any case, however, the surface of the cosmetic product is evenly covered with such grooves since the tool passes over its entire surface to suitably finish it. Of course, both in case there is a single groove and in case the grooves are uniformly distributed on the surface of the piece, they have such a small depth that cannot be seen with the naked eye, but only with a microscopic analysis. In fact, the surface is, both to the eye and to the touch, perfectly smooth and without ridges or troughs.

Various embodiments of the invention have been described but others may be conceived using the same innovative concept.

It must be said that although the above description uses a cosmetic make-up product of the baked type as an example, the device and the method may also be used with other types of cosmetic products for make-up.

1. A surface working method of a cosmetic make-up product comprising the step of setting the cosmetic make-up product in rotation about an axis of symmetry thereof and, simultaneously, moving a cutting tool above the surface of the cosmetic make-up product on a trajectory lying in a plane containing the axis of symmetry so as to remove at least a part of said surface, thus giving the surface a predetermined shape.

2. The method of claim 1, wherein the motion of the tool takes place on a single plane, and/or wherein the motion of the tool is straight, and/or wherein the tool is moved above the surface of the cosmetic make-up product at a fixed distance for each revolution of the product.

3. A device for surface working of a cosmetic make-up product, comprising a first rotatable base about a rotation axis provided with a holder for said cosmetic make-up product, and with a cutting tool associated to a mover adapted to move it along a trajectory lying in a plane containing the vertical rotation axis, over the surface of the cosmetic product at least when said rotatable base is in rotation, so as to work the surface of the product by removing at least a surface part thereof.

4. The device of claim 3, wherein said holder comprises at least a channel provided with an opening positioned on the surface of said rotatable base, and connected to a vacuum source, and wherein the opening of the channel is associated to a seal for coupling with said cosmetic make-up product.

5. The device of claim 3, wherein a second rotatable base is present for blowing a worked cosmetic product and/or for a second working thereof and/or wherein the rotation of the first base is synchronised with the rotation of the second base.

6. The device of claim 3, wherein a loading conveyor belt and an unloading conveyor belt are present, as well as a manipulator able to move the cosmetic product at least between the loading conveyor belt, the first rotating base and the unloading conveyor.

7. The device of claim 6, wherein said manipulator comprises three handlers, each fixed to a common cross member and comprising three mobile fingers for gripping the cosmetic make-up product.

8. The device of claim 7, wherein the loading conveyor belt is associated to a loading plane provided with windows enabling the mobile fingers to grip the cosmetic product.

9. The device of claim 3 wherein an an aspirator is provided, arranged above the first rotatable base, so as to aspirate cosmetic dust produced during the working.

10. A cosmetic product exhibiting, on a surface thereof, a groove having a peak-to-trough depth comprised between 30 and 60 micron, and in which such a groove is spiral-shaped, and wherein the surface of the cosmetic product is homogeneously covered with such grooves.
